(19) United States
(12) Patent Application Publication
(10) Pub. No.: US 2025/0296917 A1
(43) Pub. Date: Sep. 25, 2025

(54) SPIRO-CYCLIC AMINE DERIVATIVES AS S1P MODULATORS

(71) Applicant: AbbVie Deutschland GmbH & Co. KG, Wiesbaden (DE)

(72) Inventors: Willie AMBERG, Ludwigshafen am Rhein (DE); Gisela BACKFISCH, Eberbach (DE); Karen BODIÉ, Ludwigshafen am Rhein (DE); Katja HEMPEL, Schifferstadt (DE); Loic LAPLANCHE, Ludwigshafen am Rhein (DE); Mario MEZLER, Ludwigshafen am Rhein (DE); Michael OCHSE, Ludwigshafen am Rhein (DE); Sean TURNER, Ludwigshafen am Rhein (DE); Jeroen VAN BERGEIJK, Ludwigshafen am Rhein (DE); Elizabeth VAN DER KAM, Hamburg (DE); Udo Lange, Ludwigshafen (DE); Hervé Geneste, Ludwigshafen (DE)

(21) Appl. No.: 18/948,217

(22) Filed: Nov. 14, 2024

Related U.S. Application Data
(63) Continuation of application No. 18/443,019, filed on Feb. 15, 2024, now abandoned.
Application No. 18/443,019 is a continuation of application No. 18/342,226, filed on Jun. 27, 2023, now abandoned.
Application No. 18/342,226 is a continuation of application No. 17/537,600, filed on Nov. 30, 2021, now abandoned.
(60) Provisional application No. 63/119,690, filed on Dec. 1, 2020.

(51) Int. Cl. C07D 265/34

(52) U.S. Cl. CPC C07D 265/34

(57) ABSTRACT

The present invention is directed to spiro-cyclic amine derivatives which are modulators of S1P receptors and are useful in the treatment of CNS disorders.

### CROSS-REFERENCE TO RELATED APPLICATIONS

This application is a continuation of U.S. patent application Ser. No. 18/443,019, filed Feb. 15. 2024, which is a continuation of U.S. patent application Ser. No. 18/342,226, filed Jun. 27, 2023, which is a continuation of U.S. patent application Ser. No. 17/537,600, filed Nov. 30, 2021, which claims the benefit of U.S. Provisional Application No. 63/119,690, filed Dec. 1, 2020, which are incorporated herein by reference in their entirety and for all purposes.

### FIELD OF THE INVENTION

This invention relates to spiro-cyclic amine derivatives having affinity to S1P receptors, a pharmaceutical composition containing said compounds, as well as the use of said compounds for the preparation of a medicament for treating, alleviating or preventing diseases and conditions in which any S1P receptor is involved or in which modulation of the endogenous S1P signaling system via any S1P receptor is involved.

### BACKGROUND OF THE INVENTION

Sphingosine-1-phosphate (S1P) is a bioactive sphingolipid that mediates a wide variety of cellular responses, such as proliferation, cytoskeletal organization and migration, adherence—and tight junction assembly, and morphogenesis. S1P can bind with members of the endothelial cell differentiation gene family (EDG receptors) of plasma membrane-localized G protein-coupled receptors. To date, five members of this family have been identified as S1P receptors in different cell types, S1P1 (EDG-1), S1P2 (EDG-5), S1P3 (EDG-3), S1P4 (EDG-6) and S1P5 (EDG-8). S1P can produce cytoskeletal re-arrangements in many cell types to regulate immune cell trafficking, vascular homeostasis and cell communication in the central nervous system (CNS) and in peripheral organ systems.

It is known that S1P is secreted by vascular endothelium and is present in blood at concentrations of 200-900 nanomolar and is bound by albumin and other plasma proteins. This provides both a stable reservoir in extracellular fluids and efficient delivery to high-affinity cell-surface receptors. S1P binds with low nanomolar affinity to the five receptors S1P1-5. In addition, platelets also contain S1P and may be locally released to cause e.g. vasoconstriction. The receptor subtypes S1P1, S1P2 and S1P3 are widely expressed and represent dominant receptors in the cardiovascular system. Further, S1P1 is also a receptor on lymphocytes. S1P4 receptors are almost exclusively in the haematopoietic and lymphoid system. S1P5 is primarily (though not exclusively) expressed in central nervous system. The expression of S1P5 appears to be restricted to oligodendrocytes in mice, the myelinating cells of the brain, while in rat and man expression at the level of astrocytes and endothelial cells was found but not on oligodendrocytes.

S1P receptor modulators are compounds which signal as (ant)agonists at one or more S1P receptors. The present invention relates to modulators of the S1P5 receptor, in particular agonists, and preferably to agonists with selectivity over S1P1 and/or S1P3 receptors, in view of unwanted cardiovascular and/or immunomodulatory effects. It has now been found that S1P5 agonists can be used in the treatment of cognitive disorders, in particular age-related cognitive decline.

Although research is ongoing to develop therapeutics that can be used to treat age related cognitive decline and dementia, this has not yet resulted in many successful candidates. Therefore, there is a need for new therapeutics with the desired properties.

### SUMMARY OF THE INVENTION

The present invention provides Compound 1 having the structure:

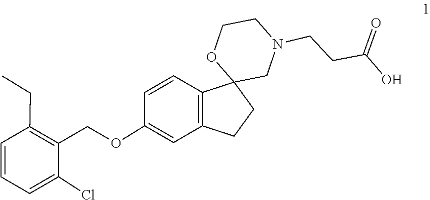 [SMILES: CCc1cccc(Cl)c1COc1ccc2c(c1)CCC21CN(CCC(=O)O)CCO1]

or a pharmaceutically acceptable salt thereof.

The present invention further provides a pharmaceutical composition comprising Compound 1, or a pharmaceutically acceptable salt thereof, and at least one pharmaceutically acceptable carrier.

The present invention further provides a method of modulating S1P receptor (e.g., S1P5) activity, comprising contacting Compound 1, or a pharmaceutically acceptable salt thereof, with an S1P receptor.

The present invention further provides a method of treating a disease or disorder associated with activity of S1P5, comprising administering to a patient in need thereof a therapeutically effective amount of Compound 1, or a pharmaceutically acceptable salt thereof.

The present invention further provides a method for treating a CNS disorder in a patient, comprising: administering to the patient a therapeutically effective amount of Compound 1, or a pharmaceutically acceptable salt thereof.

The present invention further provides use of Compound 1, or a pharmaceutically acceptable salt thereof, in therapy.

The present invention further provides Compound 1, or a pharmaceutically acceptable salt thereof, for use in the preparation of a medicament for use in therapy.

The present invention further provides a process for preparing Compound 1, or a pharmaceutically acceptable salt thereof.

### DETAILED DESCRIPTION

### Compounds

Provided herein is a compound, which is Compound 1 having the structure:

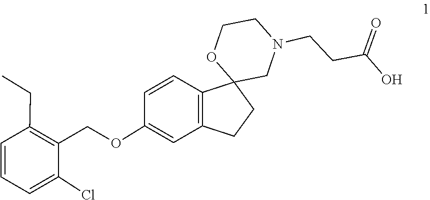 [SMILES: CCc1cccc(Cl)c1COc1ccc2c(c1)CCC21CN(CCC(=O)O)CCO1]

or a pharmaceutically acceptable salt thereof.

In some embodiments, the compound is Compound 1a having the structure:

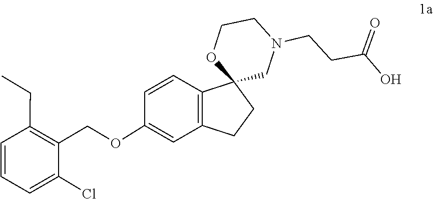

or a pharmaceutically acceptable salt thereof.

In some embodiments, the compound is Compound 1b having the structure:

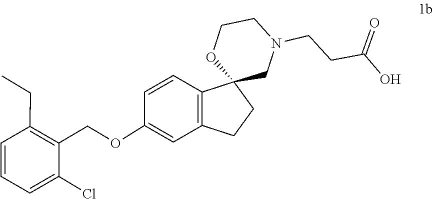

or a pharmaceutically acceptable salt thereof.

In some embodiments, the compound is 3-(5-((2-chloro-6-ethylbenzyl)oxy)-2,3-dihydrospiro[indene-1,2′-morpholin]-4′-yl)propanoic acid.

In some embodiments, the compound is (S)-3-(5-((2-chloro-6-ethylbenzyl)oxy)-2,3-dihydrospiro[indene-1,2′-morpholin]-4′-yl)propanoic acid.

In some embodiments, the compound is (R)-3-(5-((2-chloro-6-ethylbenzyl)oxy)-2,3-dihydrospiro[indene-1,2′-morpholin]-4′-yl)propanoic acid.

In some embodiment, the compound is a hydrochloric acid salt of any of the aforementioned compounds.

It is further appreciated that certain features of the invention, which are, for clarity, described in the context of separate embodiments, can also be provided in combination in a single embodiment (while the embodiments are intended to be combined as if written in multiply dependent form). Conversely, various features of the invention which are, for brevity, described in the context of a single embodiment, can also be provided separately or in any suitable subcombination.

The compounds described herein can be asymmetric (e.g., having one or more stereocenters). All stereoisomers, such as enantiomers and diastereomers, are intended unless otherwise indicated. Compounds of the present invention that contain asymmetrically substituted carbon atoms can be isolated in optically active or racemic forms. Methods on how to prepare optically active forms from optically inactive starting materials are known in the art, such as by resolution of racemic mixtures or by stereoselective synthesis.

Resolution of racemic mixtures of compounds can be carried out by any of numerous methods known in the art. One method includes fractional recrystallization using a chiral resolving acid which is an optically active, salt-forming organic acid. Suitable resolving agents for fractional recrystallization methods are, e.g., optically active acids, such as the D and L forms of tartaric acid, diacetyltartaric acid, dibenzoyltartaric acid, mandelic acid, malic acid, lactic acid or the various optically active camphorsulfonic acids such as β-camphorsulfonic acid. Other resolving agents suitable for fractional crystallization methods include stereoisomerically pure forms of α-methylbenzylamine (e.g., S and R forms, or diastereomerically pure forms), 2-phenylglycinol, norephedrine, ephedrine, N-methylephedrine, cyclohexylethylamine, 1,2-diaminocyclohexane and the like.

Resolution of racemic mixtures can also be carried out by elution on a column packed with an optically active resolving agent (e.g., dinitrobenzoylphenylglycine) using a suitable elution solvent composition.

In some embodiments, the compounds of the invention have the (R)-configuration. In other embodiments, the compounds have the (S)-configuration.

Compounds of the invention can also include all isotopes of atoms occurring in the intermediates or final compounds. Isotopes include those atoms having the same atomic number but different mass numbers. For example, isotopes of hydrogen include tritium and deuterium. One or more constituent atoms of the compounds of the invention can be replaced or substituted with isotopes of the atoms in natural or non-natural abundance. In some embodiments, the compound includes at least one deuterium atom. For example, one or more hydrogen atoms in a compound of the present disclosure can be replaced or substituted by deuterium. In some embodiments, the compound includes two or more deuterium atoms. In some embodiments, the compound includes 1, 2, 3, 4, 5, 6, 7, 8, 9, 10, 11 or 12 deuterium atoms. Synthetic methods for including isotopes into organic compounds are known in the art (Deuterium Labeling in Organic Chemistry by Alan F. Thomas (New York, N.Y., Appleton-Century-Crofts, 1971; The Renaissance of H/D Exchange by Jens Atzrodt, Volker Derdau, Thorsten Fey and Jochen Zimmermann, Angew. Chem. Int. Ed. 2007, 7744-7765; The Organic Chemistry of Isotopic Labelling by James R. Hanson, Royal Society of Chemistry, 2011). Isotopically labeled compounds can used in various studies such as NMR spectroscopy, metabolism experiments, and/or assays.

The term, “compound,” as used herein is meant to include all stereoisomers, geometric isomers, tautomers and isotopes of the structures depicted. The term is also meant to refer to compounds of the inventions, regardless of how they are prepared, e.g., synthetically, through biological process (e.g., metabolism or enzyme conversion), or a combination thereof.

All compounds, and pharmaceutically acceptable salts thereof, can be found together with other substances such as water and solvents (e.g., hydrates and solvates) or can be isolated. When in the solid state, the compounds described herein and salts thereof may occur in various forms and may, e.g., take the form of solvates, including hydrates. The compounds may be in any solid state form, such as a polymorph or solvate, so unless clearly indicated otherwise, reference in the specification to compounds and salts thereof should be understood as encompassing any solid state form of the compound.

In some embodiments, the compounds of the invention, or salts thereof, are substantially isolated. By “substantially isolated” is meant that the compound is at least partially or substantially separated from the environment in which it was formed or detected. Partial separation can include, e.g., a composition enriched in the compounds of the invention. Substantial separation can include compositions containing at least about 50%, at least about 60%, at least about 70%, at least about 80%, at least about 90%, at least about 95%, at least about 97%, or at least about 99% by weight of the compounds of the invention, or salt thereof.

The phrase “pharmaceutically acceptable” is employed herein to refer to those compounds, materials, compositions and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The expressions, “ambient temperature” and “room temperature,” as used herein, are understood in the art, and refer generally to a temperature, e.g., a reaction temperature, that is about the temperature of the room in which the reaction is carried out, e.g., a temperature from about 20° C. to about 30° C.

The present invention also includes pharmaceutically acceptable salts of the compounds described herein. The term “pharmaceutically acceptable salts” refers to derivatives of the disclosed compounds wherein the parent compound is modified by converting an existing acid or base moiety to its salt form. Examples of pharmaceutically acceptable salts include, but are not limited to, mineral or organic acid salts of basic residues such as amines; alkali or organic salts of acidic residues such as carboxylic acids; and the like. The pharmaceutically acceptable salts of the present invention can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, non-aqueous media like ether, ethyl acetate, alcohols (e.g., methanol, ethanol, iso-propanol or butanol) or acetonitrile (MeCN) are preferred. Lists of suitable salts are found in Remington's Pharmaceutical Sciences, 17^{th} Ed., (Mack Publishing Company, Easton, 1985), p. 1418, Berge et al., J. Pharm. Sci., 1977, 66(1), 1-19 and in Stahl et al., Handbook of Pharmaceutical Salts: Properties, Selection, and Use, (Wiley, 2002).

### Synthesis

Compounds of the invention, including salts thereof, can be prepared using known organic synthesis techniques and can be synthesized according to any of numerous possible synthetic routes, such as those in the Schemes below.

The reactions for preparing compounds of the invention can be carried out in suitable solvents which can be readily selected by one of skill in the art of organic synthesis. Suitable solvents can be substantially non-reactive with the starting materials (reactants), the intermediates or products at the temperatures at which the reactions are carried out, e.g., temperatures which can range from the solvent's freezing temperature to the solvent's boiling temperature. A given reaction can be carried out in one solvent or a mixture of more than one solvent. Depending on the particular reaction step, suitable solvents for a particular reaction step can be selected by the skilled artisan.

Preparation of compounds of the invention can involve the protection and deprotection of various chemical groups. The need for protection and deprotection, and the selection of appropriate protecting groups, can be readily determined by one skilled in the art. The chemistry of protecting groups is described, e.g., in Kocienski, Protecting Groups, (Thieme, 2007); Robertson, Protecting Group Chemistry, (Oxford University Press, 2000); Smith et al., March's Advanced Organic Chemistry: Reactions, Mechanisms, and Structure, 6th Ed. (Wiley, 2007); Peturssion et al., “Protecting Groups in Carbohydrate Chemistry,” J. Chem. Educ., 1997, 74(11), 1297; and Wuts et al., Protective Groups in Organic Synthesis, 4th Ed., (Wiley, 2006).

Reactions can be monitored according to any suitable method known in the art. For example, product formation can be monitored by spectroscopic means, such as nuclear magnetic resonance spectroscopy (e.g., ¹H or ¹³C), infrared spectroscopy, spectrophotometry (e.g., UV-visible), mass spectrometry or by chromatographic methods such as high performance liquid chromatography (HPLC) or thin layer chromatography (TLC).

The Schemes below provide general guidance in connection with preparing the compounds of the invention. One skilled in the art would understand that the preparations shown in the Schemes can be modified or optimized using general knowledge of organic chemistry to prepare various compounds of the invention.

Compounds 1, 1a, and 1b can be prepared, e.g., using a process as illustrated in the Schemes below.

Compounds D, E and F can be prepared using a process as illustrated in Scheme 1. In the process depicted in Scheme 1, ketone A is treated with a sulfonium salt in the presence of a base (e.g., KOH) to provide epoxide B. Epoxide B is treated with 1,2-ethanolamine in the presence of base (e.g., triethylamine) to provide diol C. Diol C is cyclized in the presence of acid (e.g., HBr in acetic acid) to provide morpholine D. Morpholine D can be resolved into enantiomers E and F by chiral resolution (e.g., supercritical fluid chromatography).

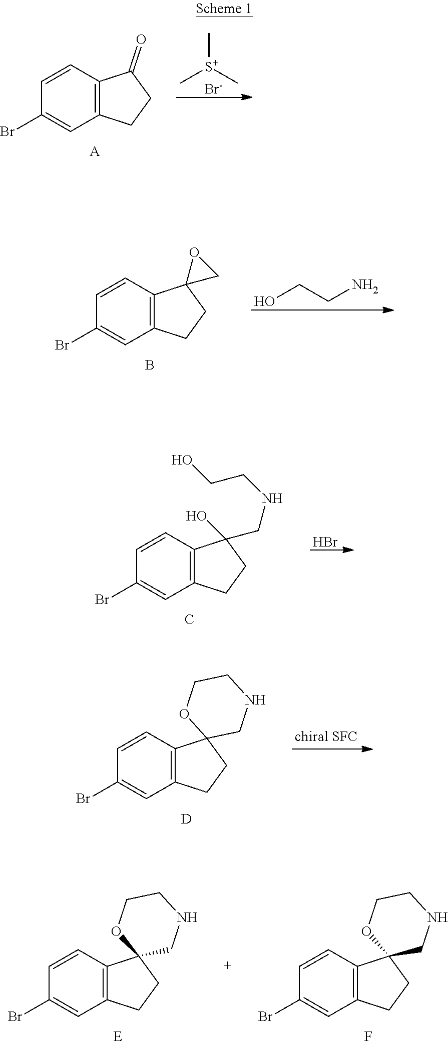

Compound 1 can be prepared using a process as illustrated in Scheme 2. Compound D is treated with ethyl acrylate to in the presence of a base (e.g., N,N-diisopropylethylamine) to provide compound G. Compound G can be coupled with (2-chloro-6-ethylphenyl)methanol under appropriate transition metal coupling conditions (e.g., a Pd catalyzed coupling reaction) to provide Compound H. For example, the coupling can be accomplished with Pd₂dba₃ in the presence of a base (e.g., cesium carbonate). Compound H can be saponified (e.g., with LiOH) and treated with acid (e.g., HCl) to provide Compound 1.

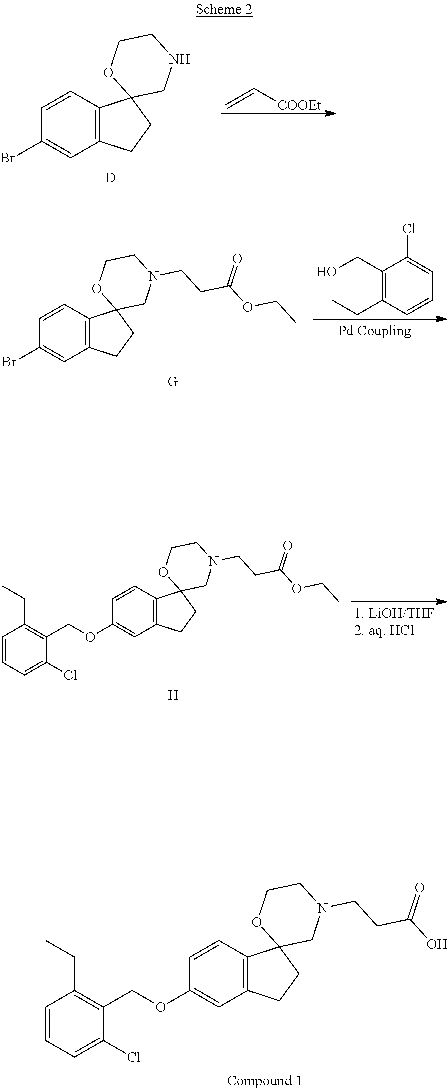

Compounds 1a and 1b can be prepared in an analgous fashion as Compound 1 according to Scheme 2 above, beginning with either Compound E or Compound F instead of Compound D, to provide Compound la or Compound 1b, respectively.

In some embodiments, the invention is directed to a process of preparing Compound 1 having the structure:

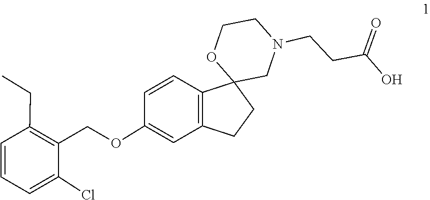 [SMILES: CCc1cccc(Cl)c1COc1ccc2c(c1)CCC21CN(CCC(=O)O)CCO1]

or a pharmaceutically acceptable salt thereof, comprising:
(a) reacting Compound G, or a salt thereof:

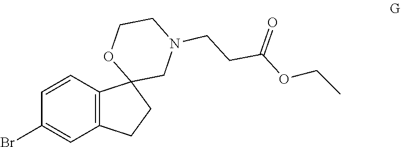 [SMILES: CCOC(=O)CCN1CCOC2(CCc3cc(Br)ccc32)C1]

with a compound having the structure:

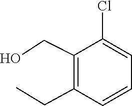

to prepare Compound H, or a salt thereof:

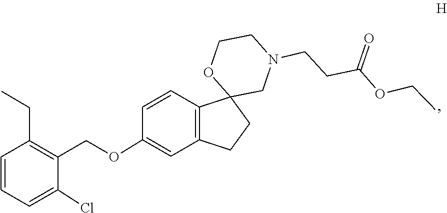 [SMILES: CCOC(=O)CCN1CCOC2(CCc3cc(OCc4c(Cl)cccc4CC)ccc32)C1]

and
(b) reacting Compound H, or a salt thereof, with hydroxide followed by treatment with an acid to form Compound 1.

In some embodiments, Compound G, or a salt thereof, is prepared by reacting Compound D, or a salt thereof:

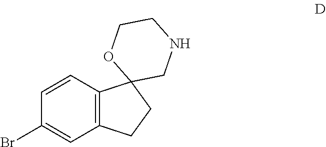 [SMILES: Brc1ccc2c(c1)CCC21CNCCO1]

with CH₂═CH—C(O)OEt.

In some embodiments, Compound D is prepared by reacting Compound C, or a salt thereof:

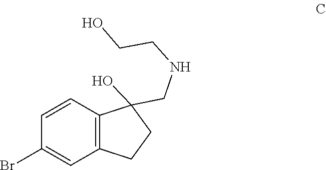 [SMILES: OCCNCC1(O)CCc2cc(Br)ccc21]

with a protic acid.

In some embodiments, Compound C, or a salt thereof, is prepared by reacting Compound B:

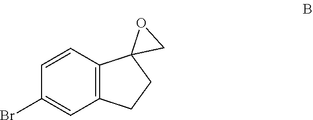 [SMILES: Brc1ccc2c(c1)CCC21CO1]

with 1,2-ethanolamine.

In some embodiments, Compound B is prepared by reacting Compound A:

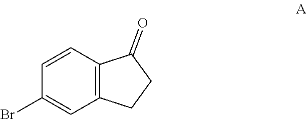 [SMILES: O=C1CCc2cc(Br)ccc21]

with trimethylsulfonium bromide.

The present invention further relates to a process for preparing a hydrochloric acid salt of Compound 1 comprising treating Compound 1 with hydrochloric acid.

The present invention further relates to a process for preparing a hydrochloric acid salt of Compound 1a comprising treating Compound la with hydrochloric acid.

The present invention further relates to a process for preparing a hydrochloric acid salt of Compound 1b comprising treating Compound 1b with hydrochloric acid.

### Methods of Treatment

Compound 1, 1a, or 1b modulates the S1P receptor, in particular the S1P5 receptor. More specifically, Compound 1, 1a, or 1b is a S1P5 receptor agonist. Compound 1, 1a, or 1b can be a selective agonist of the S1P5 receptor, wherein in the selectivity is with respect to other S1P receptors such as one or more of S1P1, S1P2, S1P3, and S1P4. Accordingly, the present invention is directed to methods of modulating the S1P5 receptor by contacting Compound 1, 1a, or 1b with the S1P5 receptor. The contacting can be carried out in vitro or in vivo.

Compound 1, 1a, or 1b (including salts thereof) is useful for treating and preventing diseases associated with S1P receptors (e.g., S1P5) or in which modulation of the endogenous S1P signaling system via any S1P receptor is involved. In particular, Compound 1, 1a, or 1b may be used to treat or prevent CNS (central nervous system) disorders, such as neurodegenerative disorders, in particular, but not limited to, cognitive disorders (in particular age-related cognitive decline) and related conditions such as, e.g., Alzheimer's disease, (vascular) dementia, Niemann-Pick disease, and cognitive deficits in schizophrenia, obsessive-compulsive behavior, major depression, autism, multiple sclerosis and pain. Preferably, the compounds of the present invention may be used to treat or prevent cognitive disorders (in particular age-related cognitive decline) and related conditions. In some embodiments, the disease is Niemann-Pick disease, such as Niemann-Pick type C.

As used herein, the term “contacting” refers to the bringing together of the indicated moieties in an in vitro system or an in vivo system such that they are in sufficient physical proximity to interact.

The terms “individual” or “patient,” used interchangeably, refer to any animal, including mammals, preferably mice, rats, other rodents, rabbits, dogs, cats, swine, cattle, sheep, horses, or primates, and most preferably humans.

The phrase “therapeutically effective amount” refers to the amount of active compound or pharmaceutical agent that elicits the biological or medicinal response in a tissue, system, animal, individual or human that is being sought by a researcher, veterinarian, medical doctor or other clinician.

As used herein, the term “treating” or “treatment” refers to one or more of (1) inhibiting the disease; e.g., inhibiting a disease, condition or disorder in an individual who is experiencing or displaying the pathology or symptomatology of the disease, condition or disorder (i.e., arresting further development of the pathology and/or symptomatology); and (2) ameliorating the disease; e.g., ameliorating a disease, condition or disorder in an individual who is experiencing or displaying the pathology or symptomatology of the disease, condition or disorder (i.e., reversing the pathology and/or symptomatology) such as decreasing the severity of disease.

In some embodiments, the compounds of the invention are useful in preventing or reducing the risk of developing any of the diseases referred to herein; e.g., preventing or reducing the risk of developing a disease, condition or disorder in an individual who may be predisposed to the disease, condition or disorder but does not yet experience or display the pathology or symptomatology of the disease.

### Combination Therapies

One or more additional pharmaceutical agents can be used in combination with Compound 1, 1a, or 1b for treatment of S1P receptor-associated diseases, disorders, or conditions described herein. The agents can be combined with the present compounds in a single dosage form, or the agents can be administered simultaneously or sequentially as separate dosage forms. In some embodiments, the additional pharmaceutical agent is an anti-Alzheimer's drug. In some embodiments, the additional pharmaceutical agent is an anti-vascular dementia drug. In some embodiments, the additional pharmaceutical agent is a cholinesterase inhibitor (e.g., donepezil, galantamine, and rivastigmine), N-methyl-D-aspartate receptor antagonist, memantine, nimodipine, hydergine, nicergoline, CDP-choline, or folic acid.

In some embodiments, the additional pharmaceutical agent is an anti-psychotic. In some embodiments, the additional pharmaceutical agent is chlorpromazine, fluphenazine, haloperidol, perphenazine, aripiprazole, asenapine, brexpiprazole, cariprazine, clozapine, lloperidone, lurasidone, olanzapine, paliperidone, quetiapine, risperidone, or ziprasidone.

### Formulations, Dosage Forms, and Administration

When employed as pharmaceuticals, the compounds of the present disclosure can be administered in the form of pharmaceutical compositions. Thus the present disclosure provides a composition comprising a compound as described herein, a compound as recited in any of the claims and described herein, or a pharmaceutically acceptable salt thereof, or any of the embodiments thereof, and at least one pharmaceutically acceptable carrier. These compositions can be prepared in a manner well known in the pharmaceutical arts, and can be administered by a variety of routes, depending upon whether local or systemic treatment is indicated and upon the area to be treated. Administration may be topical (including transdermal, epidermal, ophthalmic and to mucous membranes including intranasal, vaginal and rectal delivery), pulmonary (e.g., by inhalation or insufflation of powders or aerosols, including by nebulizer; intratracheal or intranasal), oral or parenteral. Parenteral administration includes intravenous, intraarterial, subcutaneous, intraperitoneal intramuscular or injection or infusion; or intracranial, e.g., intrathecal or intraventricular, administration. Parenteral administration can be in the form of a single bolus dose, or may be, e.g., by a continuous perfusion pump. Pharmaceutical compositions and formulations for topical administration may include transdermal patches, ointments, lotions, creams, gels, drops, suppositories, sprays, liquids and powders. Conventional pharmaceutical carriers, aqueous, powder or oily bases, thickeners and the like may be necessary or desirable.

This invention also includes pharmaceutical compositions which contain, as the active ingredient, the compound of the present disclosure or a pharmaceutically acceptable salt thereof, in combination with one or more pharmaceutically acceptable carriers. In some embodiments, the composition is suitable for topical administration. In making the compositions of the invention, the active ingredient is typically mixed with an excipient, diluted by an excipient or enclosed within such a carrier in the form of, e.g., a capsule, sachet, paper, or other container. When the excipient serves as a diluent, it can be a solid, semi-solid, or liquid material, which acts as a vehicle, carrier or medium for the active ingredient. Thus, the compositions can be in the form of tablets, pills, powders, lozenges, sachets, cachets, elixirs, suspensions, emulsions, solutions, syrups, aerosols (as a solid or in a liquid medium), ointments containing, e.g., up to 10% by weight of the active compound, soft and hard gelatin capsules, suppositories, sterile injectable solutions and sterile packaged powders.

In some embodiments, the composition is a sustained release composition comprising at least one compound described herein, or a pharmaceutically acceptable salt thereof, and at least one pharmaceutically acceptable carrier or excipient

The compositions can be formulated in a unit dosage form, each dosage containing from about 5 to about 1,000 mg (1 g). The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient.

The active compound may be effective over a wide dosage range and is generally administered in a therapeutically effective amount. It will be understood, however, that the amount of the compound actually administered will usually be determined by a physician, according to the relevant circumstances, including the condition to be treated, the chosen route of administration, the actual compound administered, the age, weight, and response of the individual patient, the severity of the patient's symptoms and the like.

The therapeutic dosage of a compound of the present invention can vary according to, e.g., the particular use for which the treatment is made, the manner of administration of the compound, the health and condition of the patient, and the judgment of the prescribing physician. The proportion or concentration of a compound of the invention in a pharmaceutical composition can vary depending upon a number of factors including dosage, chemical characteristics (e.g., hydrophobicity), and the route of administration. The dosage is likely to depend on such variables as the type and extent of progression of the disease or disorder, the overall health status of the particular patient, the relative biological efficacy of the compound selected, formulation of the excipient, and its route of administration. Effective doses can be extrapolated from dose-response curves derived from in vitro or animal model test systems.

The liquid forms in which the compounds and compositions of the present invention can be incorporated for administration orally or by injection include aqueous solutions, suitably flavored syrups, aqueous or oil suspensions, and flavored emulsions with edible oils such as cottonseed oil, sesame oil, coconut oil, or peanut oil, as well as elixirs and similar pharmaceutical vehicles.

Compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as described supra. In some embodiments, the compositions are administered by the oral or nasal respiratory route for local or systemic effect. Compositions can be nebulized by use of inert gases. Nebulized solutions may be breathed directly from the nebulizing device or the nebulizing device can be attached to a face mask, tent, or intermittent positive pressure breathing machine. Solution, suspension, or powder compositions can be administered orally or nasally from devices which deliver the formulation in an appropriate manner.

Topical formulations can contain one or more conventional carriers. In some embodiments, ointments can contain water and one or more hydrophobic carriers.

### EXAMPLES

Experimental procedures for compounds of the invention are provided below. Where the preparation of starting materials is not described, these are commercially available, known in the literature, or readily obtainable by those skilled in the art using standard procedures. All solvents used were commercially available and were used without further purification. Reactions were typically run using anhydrous solvents under an inert atmosphere of nitrogen.

### Example 1. Synthesis of (S)-3-(5-((2-chloro-6-ethylbenzyl)oxy)-2,3-dihydrospiro[indene-1,2′-morpholin]-4′-yl)propanoic acid (Compound 1a)

Step 1. 5-bromo-2,3-dihydrospiro[indene-1,2′-oxirane]

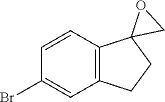 [SMILES: Brc1ccc2c(c1)CCC21CO1]

To a solution of trimethylsulfonium bromide (726 g, 4.62 mol) in acetonitrile (4.7 L) and H₂O (345 mL) was added KOH (1.04 kg, 18.5 mol) at 55° C. for 10 min. 5-Bromo-2,3-dihydro-1H-inden-1-one (650 g, 3.08 mol) was added to the reaction mixture and stirred at 55° C. for 20 min. This reaction was carried out in triplicate in parallel. The crude reaction mixtures were cooled to 15° C., combined, and extracted with petroleum ether: TBME (4:1, 10 6 L×4). A small sample of the crude product was concentrated in vacuo and analyzed by ¹H NMR to confirm the identity of the intended product, 5-bromo-2,3-dihydrospiro[indene-1,2′-oxirane]. ¹H NMR (400 MHz, DMSO-d₆) δ 7.47 (s, 1H), 7.36 (d, J=8.0 Hz, 1H), 6.96 (d, J=8.0 Hz, 1H), 3.18-3.25 (m, 2H), 2.93-3.05 (m, 2H), 2.37-2.38 (m, 1H), 2.01-2.12 (m, 1H).

Step 2. 5-bromo-1-(((2-hydroxyethyl)amino)methyl)-2,3-dihydro-1H-inden-1-ol

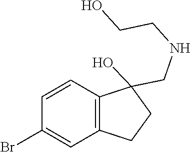 [SMILES: OCCNCC1(O)CCc2cc(Br)ccc21]

The organic layer was divided into four equal portions for this reaction step. To a solution of 1,2-ethanolamine (706 g, 11.6 mol) and triethylamine (1.17 kg, 11.6 mol) in isopropyl alcohol (2.0 L) was added the solution of crude 5-bromo-2,3-dihydrospiro[indene-1,2′-oxirane] from Step 1 (2.31 mol) in one portion, and the mixture was stirred at 50° C. for 5 h. The four reaction mixtures were combined, then concentrated in vacuo. The residue was dissolved in ethyl acetate (8 L), washed successively with a saturated solution saturated of Na₂CO₃ (4 L), water (4 L), and brine (2 L). The organic layer was separated and concentrated in vacuo to afford 5-bromo-1-(((2-hydroxyethyl)amino)methyl)-2,3-dihydro-1H-inden-1-ol (2.00 kg) as a brown oil. ¹H NMR (400 MHz, DMSO-d₆) δ 7.29-7.36 (m, 2H), 7.24 (d, J=8.0 Hz, 1H), 3.37-3.41 (m, 2H), 2.63-2.72 (m, 1H), 2.57-2.60 (m, 3H), 2.52-2.53 (m, 2H), 2.23-2.31 (m, 1H), 1.85-1.88 (m, 1H).

Step 3. 5-bromo-2,3-dihydrospiro[indene-1,2′-morpholine]

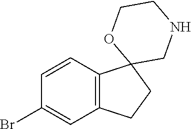 [SMILES: Brc1ccc2c(c1)CCC21CNCCO1]

To a solution of hydrogen bromide in acetic acid (735 g, 40%) and water (4.50 L) was added the crude 5-bromo-1-(((2-hydroxyethyl)amino)methyl)-2,3-dihydro-1H-inden-1-ol (650 g, 2.27 mol) from Step 2, and the mixture was stirred at 80° C. for 12 h. After cooling to 15° C., the three reaction mixtures were combined and filtered. The filter cake was dissolved in ethyl alcohol: H₂O (10 L, 4:1) at 80° C. The mixture was cooled to room temperature. After 3 days, the crystallized product was filtered. The filter cake was dissolved in water (3 L), and the pH of the mixture was adjusted to 8 by addition of sodium carbonate. The mixture was extracted with ethyl acetate (2×2 L). Activated carbon (200 g) was added to the organic layer, and the mixture was stirred for 2 h. The resulting solution was filtered, and the filtrate was concentrated to afford 5-bromo-2,3-dihydrospiro[indene-1,2′-morpholine] (770 g) as an off-white solid.

¹H NMR (400 MHz, CDC13) 8 7.34-7.41 (m, 3H), 3.76-3.78 (m, 2H), 2.99-3.02 (m, 2H), 2.94-2.98 (m, 2H), 2.77-2.83 (m, 2H), 2.51-2.52 (m, 1H), 2.17-2.20 (m, 1H), 1.69 (br. s, 1H)

Step 4. (S)-5-bromo-2,3-dihydrospiro[indene-1,2′-morpholine]

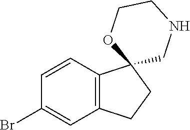 [SMILES: Brc1ccc2c(c1)CC[C@@]21CNCCO1]

The enantiomers of 5-bromo-2,3-dihydrospiro[indene-1,2′-morpholine] were separated by chiral supercritical fluid chromatography (SFC) under the following conditions:
Column: ChiralPak AY, 150×4.6 mm I.D., 3 μm
Mobile phase: A for CO₂ and B for isopropanol (with 0.05% diethylamine)
Gradient: B 5-40%, flow rate: 2.4 mL/min
Back pressure: 100 bar
Column temperature: 35° C.
UV detector: 220 nm

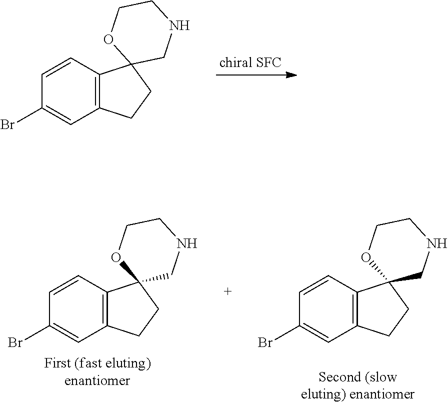

The first (fast eluting) and second (slow eluting enantiomer) enantiomer were obtained pure in 97.6% ee and 98.6% ee, respectively. The first (fast eluting) enantiomer is believed to correspond to (S)-5-bromo-2,3-dihydrospiro[indene-1,2′-morpholine], as determined by single crystal X-ray diffraction.

¹H NMR (400 MHz, CDCl₃) δ ppm 2.15-2.22 (m, 1H) 2.52 (ddd, J=12.84, 8.43, 4.08 Hz, 1H) 2.77-2.77 (m, 1H) 2.77-2.88 (m, 2H) 2.89-3.05 (m, 4H) 3.78 (dd, J=6.73, 2.98 Hz, 2H) 7.36-7.42 (m, 3H).

Step 5. (S)-ethyl 3-(5-bromo-2,3-dihydrospiro[indene-1,2′-morpholin]-4′-yl)propanoate (Compound 1a)

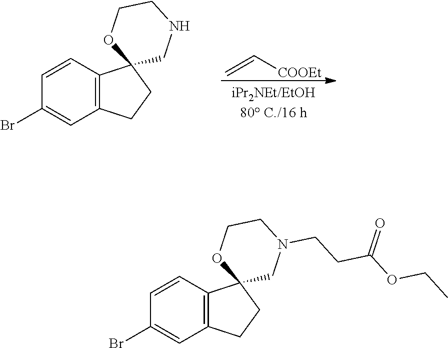

To a solution of(S)-5-bromo-2,3-dihydrospiro[indene-1,2′-morpholine] (40 g, 149 mmol) in ethanol (400 ml) was added N,N-diisopropylethylamine (8 ml, 448 mmol) and ethyl acrylate (44.8 g, 448 mmol), then the mixture was stirred at 80° C. for 16 h. The reaction mixture was concentrated and the crude product was purified by column chromatography on silica gel (petroleum ether: ethyl acetate=1:0 to 10:1) to afford (S)-ethyl 3-(5-bromo-2,3-dihydrospiro[indene-1,2′-morpholin]-4′-yl)propanoate (51 g, 138 mmol, 93% yield) as a brown oil. The product was analyzed as follows:

### LC/MS method

Column: 2.0×50 mm phenomenex Luna-C18 column, 5 μm
Mobile phase: A for 0.0375% CF₃CO₂H in water and B for 0.018% CF₃CO₂H in CH₃CN
Gradient: 10-100% B in 3.4 min with a hold at 100% B for 0.45 min, 100-10% B in 0.01 min, and then held at 10% B for 0.65 min (0.8 mL/min flow rate).
Detection methods: diode array, evaporative light scattering (ELSD), and positive electrospray ionization.

### HPLC Method

Column: 2.0×50 mm phenomenex Luna-C18 column (5 μm particles)
Mobile phase: A for 0.0375% TFA in water and B for 0.018% TFA in MeCN
Gradient: 10-80% B in 4 min, held at 80% B for 0.9 min, 80-10% B in 0.01 min, and then held at 10% B for 1 min (0.8 mL/min flow rate)
Detection method: diode array
QC: m/z=368.1 and 370.1
HPLC: product RT: 2.13 min

¹HNMR (400 MHz, CDCl₃) δ ppm 1.27 (t, J=7.17 Hz, 3H) 2.10 (ddd, J=13.29, 7.66, 6.17 Hz, 1H) 2.33-2.61 (m, 7H) 2.61-2.85 (m,3H) 2.98-3.08 (m, 1H) 3.68-3.80 (m, 2H) 4.16 (q, J=7.06 Hz, 2H) 7.31 (d, J=7.94 Hz, 1H) 7.37 (s, 1H) 7.49 (br d, J=7.94 Hz, 1H).

Step 6. (S)-ethyl 3-(5-bromo-2,3-dihydrospiro[indene-1,2′-morpholin]-4′-yl)propanoate

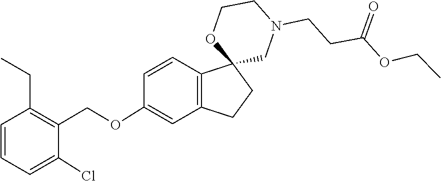 [SMILES: CCOC(=O)CCN1CCO[C@]2(CCc3cc(OCc4c(Cl)cccc4CC)ccc32)C1]

A 3-neck flask was charged with glass beads (4 mm, 165 g), Pd₂dba₃ (11.44 g, 12.49 mmol), 2-(di-tert-butylphosphino)-2′,4′,6′-triisopropyl-3,6-dimethoxy-1,1′-biphenyl (12.41 g, 25.6 mmol), and cesium carbonate (305 g, 937 mmol). The flask was equipped with an overhead agitator, reflux condenser, and a Claisen adapter with argon inlet and temperature probe. The flask was purged with argon for 90 min to remove all oxygen.

In a separate flask, (S)-ethyl 3-(5-bromo-2,3-dihydrospiro[indene-1,2′-morpholin]-4′-y1)propanoate (230 g, 625 mmol), (2-chloro-6-ethylphenyl)methanol (CAS Registry #1268862-18-5, 128 g, 749 mmol) were mixed in toluene (1840 ml) until a homogeneous solution was obtained. The mixture was purged with argon for 1 h. Using argon pressure, the solution was transferred by canula to the reaction flask. The reaction mixture was warmed to 70° C., and stirred for 14 h. The reaction mixture was cooled to room temperature using a water bath and the inorganic material and glass beads were filtered off. The reaction flask was washed with ethyl acetate (1 L) and any residual solid was filtered off. The filter cake was washed with ethyl acetate (1.3 L). The dark brown solution was washed (2×2000 g) with a freshly prepared solution of 5% L-cysteine (200 g) and 8% NaHCO₃ (320 g) in water (3480 g). The solution was allowed to settle for 25 min between washes. The organic layer was separated and filtered to remove any residual particulate.

A small sample was concentrated in vacuo and analyzed by ¹H NMR to confirm the identity of the desired product, (S)-ethyl-3-(5-((2-chloro-6-ethylbenzyl)oxy)-2,3dihydrospiro[indene-1,2′-morpholin]-4′-yl)propanoate. ¹HNMR (400 MHz, CDCl₁₃) δ ppm 7.48 (br d, J=8.19 Hz, 1H) 7.12-7.23 (m, 2H) 7.08 (m, 1H) 6.83 (s, 1H) 6.78 (dd, J=8.38, 2.02 Hz, 1H) 5.09 (s, 2H) 4.09 (q, J=7.13 Hz, 2H) 3.68 (m, 2H) 2.97 (m, 1H) 2.58-2.77 (m, 5H) 2.38-2.51 (m, 6H) 2.28 (m, 1H) 2.07 (ddd, J=13.24, 8.16, 5.38 Hz, 1H) 1.18 (m, 3H) 1.13-1.16 (m, 3H).

Step 7. (S)-3-(5-((2-Chloro-6-ethylbenzyl)oxy)-2,3-dihydrospiro[indene-1,2′-morpholin]-4′-yl)propanoic acid

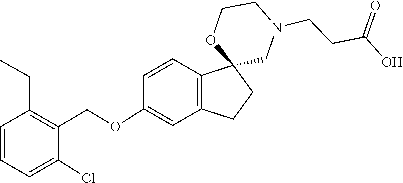 [SMILES: CCc1cccc(Cl)c1COc1ccc2c(c1)CC[C@@]21CN(CCC(=O)O)CCO1]

The crude (S)-ethyl 3-(5-((2-chloro-6-ethylbenzyl)oxy)-2,3-dihydrospiro[indene-1,2′-morpholin]-4′-yl)propanoate (4.31 g, 9.41 mmol) from Step 6 was dissolved in THF (1.6 L) solution and stirred. Lithium hydroxide (70.6 g, 2948 mmol) and 600 mL of water were added. After 20 h, the organic and aqueous layers were separated. The aqueous layer was titrated to a pH of 7 using 1M HCl. Then, the aqueous layer was extracted with ethyl acetate (3×350 mL) then with a 1:1 (v/v) of CHCl₃/isopropyl alcohol. The combined organic layers were dried via stirring for 30 min over Na₂SO₄. After filtration and removal of the solvent, the crude residue was dissolved in a minimal amount of THF. (S)-3-(5-((2-Chloro-6-ethylbenzyl)oxy)-2,3-dihydrospiro[indene-1,2′-morpholin]-4′-yl)propanoic acid was obtained by precipitation with heptane. This product was used in Step 8 without further purification.

Step 8: (S)-3-(5-((2-Chloro-6-ethylbenzyl)oxy)-2,3-dihydrospiro[indene-1,2′-morpholin]-4′-yl)propanoic acid hydrochloride

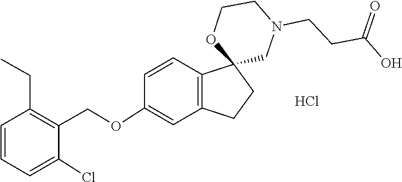

Several batches of the product from Step 7 were combined and re-crystallized as the HCl salt as follows. (S)-3-(5-((2-Chloro-6-ethylbenzyl)oxy)-2,3-dihydrospiro[indene-1,2′-morpholin]-4′-yl)propanoic acid (279 g, 649 mmol) was dissolved in ethyl acetate (2.8 L) and passed through a 0.45 micron filter to remove all particulates. The solution was cooled to 10° C. and 1.1 eq of 4M HCl in dioxane (178 mL) was added dropwise. Additional ethyl acetate was added to the thick solution to enable stirring. The solid was filtered at room temperature, washed with ethyl acetate, and dried in the vacuum oven at 45° C. overnight to provide the desired (S)-3-(5-((2-chloro-6-ethylbenzyl)oxy)-2,3-dihydrospiro[indene-1,2′-morpholin]-4′-yl)propanoic acid hydrochloride (280 g). ¹HNMR (400 MHz, DMSO-d₆) δ ppm 7.19-7.44 (m, 4H) 7.00 (s, 1H) 6.93 (d, J=8.16 Hz, 1H) 5.14 (s, 2H) 3.94 (d, J=5.07 Hz, 2H) 3.17-3.33 (m, 4H) 3.02-3.16 (m, 2H) 2.82-2.99 (m, 5H) 2.71 (q, J-7.28 Hz, 2H) 2.12 (m, 1H) 1.16 (t, J=7.50 Hz, 3H).

### Example A. In Vitro Activity

Agonist potency and efficacy were assessed by measurement of intracellular Ca230 release. Recombinant CHO-K1 cells (Euroscreen, Brussels, Belgium) expressing human S1PR5, S1PR1, S1PR3 or S1PR4 receptors, aequorin, and GTP binding protein Gq/i5 were cultured using a medium containing nutrient mixture F-12 Ham (Sigma-Aldrich) supplemented with 10% FBS, 100 pg/mL gentamicin and equilibrated at 5% C02. 15.000 cells in 20 pL medium were seeded into Biocoat poly-D-Lysine coated 384 well plates (Becton Dickinson #35-6663) and grown to ˜95% confluency after 24 h. Culture medium was replaced by an assay buffer consisting of HBBS with Ca2+ and Mg2+ (Invitrogen #14025-050), 20 mmol/L Hepes (Sigma-Aldrich #H-3375), 2.5 mmol/L probenecid (Sigma-Aldrich #P-8761, and 0.1% BSA (Sigma-Aldrich #A-7030) pH 7.4. The Calcium 5 no-wash FLIPR assay kit (Molecular Devices #5000625) was performed as described in the kit instructions. Cells were incubated with Calcium 5 dye for 1 h at 37° C., 5% CO2 in the dark. After 45 min adaptation to RT assessment of agonist stimulation of intracellular Ca2+ release was performed by addition of test compounds at various concentrations obtained by serial dilution. Phospho-fingolimod was used as positive control and reference agonist (intrinsic activity 100%). Agonism of test compounds at the human S1Px receptors were characterized by the EC50 values (potencies) deduced from a nonlinear 4 parametric logistic curve fit to the measured fluorescence data after normalization to the lower and upper plateaus of the fitted reference agonist's (phospho-fingolimod) effect curve and by their efficacy (Emax) values represented by their maximum achievable effects in % of the reference agonist's efficacy, which is defined as an Emax of 100%.

Efficacy testing for hS1PR2 was performed at Euroscreen, Eurofins, France. Cells co-expressing mitochondrial Apoaequorin, recombinant human Gα16 and S1P2 receptor (FAST-0198A) grown to mid-log phase in culture media without antibiotics were detached with PBS-EDTA, centrifuged and resuspended in assay buffer (DMEM/HAM's F12 with HEPES, without phenol red+0.1 fatty acid free BSA) at a concentration of 1×106 cells/ml. Cells were incubated at room temperature for at least 4 h with coelenterazine. The reference agonists S1P and JTE-013 were tested to evaluate the performance of the assay on each day of the test and determine the EC50. For agonist testing, cell suspension was mixed 1:1 with test or reference agonist in 384-well plate. The resulting emission of light was recorded using a Hamamatsu Functional Drug Screening System 6000 (FDSS) luminometer. On each day of experimentation, reference compounds were tested at several concentrations to obtain a dose-response curve and an estimated EC50 value. Reference values thus obtained for the test were compared to historical values obtained from the same receptor and used to validate the experimental session.

Receptor agonism data is provided in the below table.

| | hS1PR5 | | hS1PR4 | | hS1PR3 | hS1PR2 | hS1PR1 |
|---|---|---|---|---|---|---|---|
| | pEC50 | Emax | pEC50 | Emax | pEC50 | pEC50 | pEC50 |
| | [M] | [%] | [M] | [%] | [M] | [M] | [M] |
| Com- | 9.60 | 73 | 5.83 | 88 | >5.30 | >5.30 | >5.30 |
| pound | | | | | | | |
| 1a | | | | | | | |

Various modifications of the invention, in addition to those described herein, will be apparent to those skilled in the art from the foregoing description. Such modifications are also intended to fall within the scope of the appended claims. Each reference, including all patent, patent applications, and publications, cited in the present application is incorporated herein by reference in its entirety.

What is claimed is:

1. A compound, which is Compound 1 having the structure: 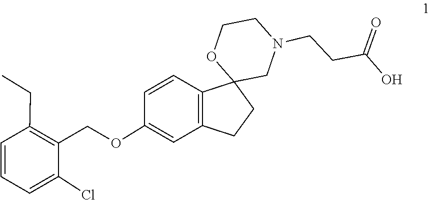 [SMILES: CCc1cccc(Cl)c1COc1ccc2c(c1)CCC21CN(CCC(=O)O)CCO1]
or a pharmaceutically acceptable salt thereof.

2. The compound of claim 1, which is Compound 1a having the structure: 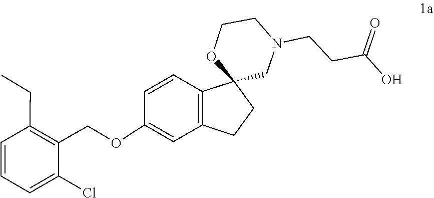
or a pharmaceutically acceptable salt thereof.

3. The compound of claim 1, which is Compound 1b having the structure: 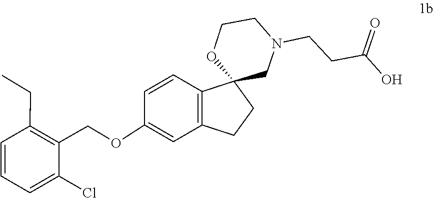
or a pharmaceutically acceptable salt thereof.

4. The compound of claim 1, which is a hydrochloric acid salt.

5. A pharmaceutical composition comprising the compound of claim 1, or a pharmaceutically acceptable salt thereof, and at least one pharmaceutically acceptable carrier.

6. A method of modulating S1P receptor activity, said method comprising contacting a compound of claim 1, or a pharmaceutically acceptable salt thereof, with an S1P receptor.

7. The method of claim 6, wherein the S1P is S1P5.

8. The method of claim 6, wherein the contacting comprises administering the compound to a patient.

9. A method of treating a disease or disorder associated with S1P5, said method comprising administering to a patient in need thereof a therapeutically effective amount of a compound of claim 1, or a pharmaceutically acceptable salt thereof.

10. A method of treating a CNS disorder in a patient in need thereof, said method comprising administering to the patient a therapeutically effective amount of the compound of claim 1, or a pharmaceutically acceptable salt thereof.

11. The method of claim 10, wherein the CNS disorder is Alzheimer's disease, or vascular dementia.

12. The method of claim 10, wherein the CNS disorder is Niemann-Pick disease.

13. The method of claim 10, wherein the CNS disorder is Niemann-Pick type C disease.

14. The method of claim 10, wherein the CNS disorder is cognitive deficits in schizophrenia, obsessive-compulsive behavior, major depression, autism, multiple sclerosis, or pain.

15. The method of claim 10, wherein the CNS disorder is a cognitive disorder.

16. The method of claim 15, wherein the cognitive disorder is age-related cognitive decline.

17. A process of preparing Compound 1 having the structure: 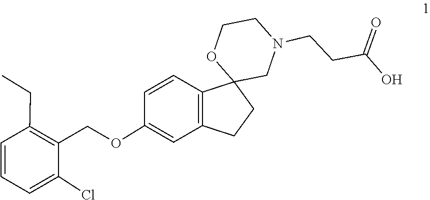 [SMILES: CCc1cccc(Cl)c1COc1ccc2c(c1)CCC21CN(CCC(=O)O)CCO1]
or a pharmaceutically acceptable salt thereof, comprising:
(a) reacting Compound G, or a salt thereof: 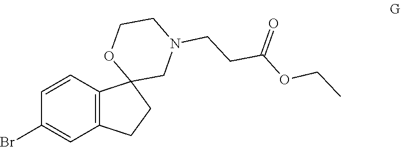 [SMILES: CCOC(=O)CCN1CCOC2(CCc3cc(Br)ccc32)C1]
with a compound having the structure: 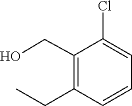
to prepare Compound H, or a salt thereof: 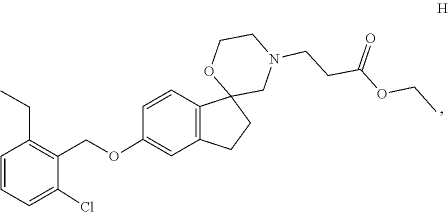 [SMILES: CCOC(=O)CCN1CCOC2(CCc3cc(OCc4c(Cl)cccc4CC)ccc32)C1]
and
(b) reacting Compound H, or a salt thereof, with hydroxide followed by treatment with an acid to form Compound 1.

18. The process of claim 17 wherein Compound G, or a salt thereof, is prepared by reacting Compound D, or a salt thereof: 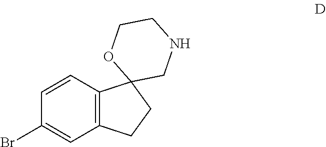 [SMILES: Brc1ccc2c(c1)CCC21CNCCO1]
with CH₂═CH—C(O)OEt.

19. The process of claim 18 wherein Compound D is prepared by reacting Compound C, or a salt thereof: 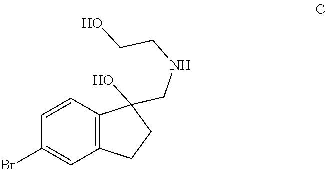 [SMILES: OCCNCC1(O)CCc2cc(Br)ccc21]
with a protic acid.

20. The process of claim 19 wherein Compound C, or a salt thereof, is prepared by reacting Compound B: 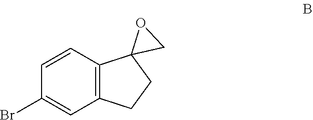 [SMILES: Brc1ccc2c(c1)CCC21CO1]
with 1,2-ethanolamine.

21. The process of claim 20 wherein Compound B is prepared by reacting Compound A: 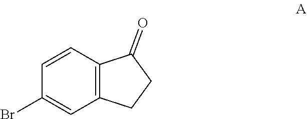 [SMILES: O=C1CCc2cc(Br)ccc21]
with trimethylsulfonium bromide.

22. A compound, which is Compound 1, prepared by the process of claim 17.
